(19) United States
(12) Patent Application Publication
(10) Pub. No.: US 2025/0388881 A1
(43) Pub. Date: Dec. 25, 2025

(54) METHOD FOR IMPROVING THERMOSTABILITY OF PHYTASE, MUTANT AND USE

(71) Applicant: INSTITUTE OF ANIMAL SCIENCE OF CHINESE ACADEMY OF AGRICULTURAL SCIENCES, BEIJING (CN)

(72) Inventors: Huoqing HUANG, BEIJING (CN); Tao TU, BEIJING (CN); Qian WANG, BEIJING (CN); Bin YAO, BEIJING (CN); Huiying LUO, BEIJING (CN); Yingguo BAI, BEIJING (CN); Yuan WANG, BEIJING (CN); Xiaoyun SU, BEIJING (CN); Yaru WANG, BEIJING (CN); Jie ZHANG, BEIJING (CN); Xing QIN, BEIJING (CN); Xiaolu WANG, BEIJING (CN); Honglian ZHANG; Huimin YU, BEIJING (CN)

(21) Appl. No.: 18/879,267

(22) PCT Filed: Jan. 30, 2023
(86) PCT No.: PCT/CN2023/073753

§ 371 Date: Dec. 27, 2024

(30) Foreign Application Priority Data
Jun. 28, 2022 (CN) 202210739640.9

(51) Int. Cl. C12N 9/16; C12P 9/00

(52) U.S. Cl. CPC C12N 9/16; C12P 9/00; C12Y 301/03026

(57) ABSTRACT

The present invention relates to the field of genetic engineering, particularly to method for improving thermo-stability of phytase, mutant and use. The present invention introduces a series of mutations to the phytase APPAmut4, which may involve introducing disulfide bonds, reducing the free energy of unfolding, optimizing the key residues in the coevolution process, and significantly improving the thermal stability of the phytase. Among the mutants of the present invention, the optimal mutant APPAmut9 retains about 70% of its activity after being treated for 5 minutes at 100° C., while the phytase APPAmut4 has already been inactivated. Therefore, the present invention overcomes the shortcomings of the prior art and provides phytase mutants with high thermal stability suitable for wide application in fields such as energy, food, and feed.

### FIELD OF THE INVENTION

The present invention relates to the field of genetic engineering, particularly to method for improving thermo-stability of phytase, mutant and use.

### BACKGROUND OF THE INVENTION

Phytase is an important industrial enzyme that can hydrolyze phytic acid into phosphoric acid residues. Phytase has been widely applied in various industries such as food processing, environmental protection, feed additive, and biofuel production. The heat resistance of phytase has become one of the bottleneck issues limiting its industrial application in the light of the requirement of the instantaneous high temperature during the process of feed granulation.

The formation of the disulfide bonds in proteins is an oxidation process that creates a covalent bond connecting the sulfur atoms of two cysteine residues, fixing the protein structure and stabilizing its active conformation, thereby contributing to its activity and stability. Therefore, the disulfide bond engineering is an effective strategy to improve the thermal stability of the protein. However, not all introduction of the disulfide bonds can improve the thermal stability. For example, Liu et al. found that when seven pairs of cysteine were introduced into alkaline α-amylase derived from Alkalinimonas amylolytica, only three pairs, P35C-G426C, G116C-Q120C, and R436C-M480C, resulted in enhanced thermal stability of the enzyme, with the half lives at 60° C. increased by 4.1, 7.4, and 2.2 minutes, respectively, as compared to that of the wild type, while the introduction of the remaining four pairs of cysteine actually reduced thermal stability (LONG et al., 2014). Therefore, how to select appropriate mutation sites to introduce disulfide bonds is a key issue in improving enzyme thermal stability. The unfolding free energy Δ Gu reflects the difference in Gibbs free energy between the folding and unfolding states of a protein, which is an important indicator of protein thermal stability. It can be observed that some amino acid residues are highly correlated with each other rather than independent in multi-sequence alignment, which is manifested as a compensatory phenomenon in the process of evolution that the related residue compensate for the adverse effect when a certain amino acid of a protein undergoes a mutation, thereby maintaining the structure and function of the protein, and also named as a coevolution phenomenon that it's very important for maintaining the stability of the protein that the coevolutionary residues with correlation are adjacent in spatial structure (SUTTO et al., 2015).

### Order of the Invention

One order of the present invention is to provide phytase mutant having the improved thermal stability of the phytase APPAmut4 derived from Yersinia intermedia.

Another order of the present invention is to provide a gene encoding the above phytase mutant.

Another order of the present invention is to provide a recombinant vector comprising the above gene encoding the above phytase mutant.

Another order of the present invention is to provide a recombinant strain comprising the above gene encoding the above phytase mutant.

Another order of the present invention is to provide a method of preparing a phytase having the improved thermal stability.

Another order of the present invention is to provide a use of the above phytase mutant.

### SUMMARY OF THE INVENTION

One aspect of the present invention is to provide a phytase mutant having the improved thermal stability by performing mutation to the phytase APPAmut4 comprising an amino acid sequence of SEQ ID NO:1, derived from Yersinia intermedia.

The present invention provides a phytase mutant having phytase activity and comprising the amino acid sequence of SEQ ID NO:1, except A corresponding to position 57 and A corresponding to position 103 of SEQ ID NO: 1 are replaced with C, and C respectively (i.e, mutant A57C/A103C), or,
G corresponding to position 101 and V corresponding to position 116 of SEQ ID NO: 1 are replaced with C, and C respectively (i.e. mutant G101C/V116C), or
R corresponding to position 271 and E corresponding to position 413 of SEQ ID NO: 1 are replaced with C, and C respectively (i.e. mutant R271C/E413C), or
R corresponding to position 353 and L corresponding to position 401 of SEQ ID NO: 1 are replaced with C, and C respectively (i.e. mutant R353C/L401C), or
A corresponding to position 147 and Y corresponding to position 268 of SEQ ID NO: 1 are replaced with C, and C respectively (i.e. mutant A147C/Y268C), and
wherein the phytase mutant has improved thermal stability as compared to a phytase consisting of the amino acid sequence of SEQ ID NO:1.

The phytase mutant according to the present invention is the phytase mutant APPAmut5 having phytase activity and comprising the amino acid sequence of SEQ ID NO: 1, except A corresponding to position 57, A corresponding to position 103, G corresponding to position 101, V corresponding to position 116, R corresponding to position 271, E corresponding to position 413, R corresponding to position 353, L corresponding to position 401, A corresponding to position 147, Y corresponding to position 268 of SEQ ID NO: 1 are replaced with C respectively, and wherein the phytase mutant has improved thermal stability as compared to a phytase consisting of the amino acid sequence of SEQ ID NO: 1.

The phytase mutant APPAmut5 has an amino acid sequence of SEQ ID NO: 2.

Further, the present invention provides the phytase mutant having phytase activity and comprising the amino acid sequence of SEQ ID NO:1, except G corresponding to position 65 is replaced with R, or,
E corresponding to position 282 is replaced with L, or,
G corresponding to position 365 is replaced with D, or,
D corresponding to position 133 is replaced with E, or,
R corresponding to position 382 is replaced with I, or,
S corresponding to position 393 is replaced with I, and wherein the phytase mutant has improved thermal stability as compared to a phytase consisting of the amino acid sequence of SEQ ID NO: 1.

The phytase mutant according to the present invention is the phytase mutant APPAmut8 having phytase activity and comprising the amino acid sequence of SEQ ID NO: 2, except G corresponding to position 65 is replaced with R, E corresponding to position 282 is replaced with L, and G corresponding to position 365 is replaced with D, and wherein the phytase mutant has improved thermal stability as compared to a phytase consisting of the amino acid sequence of SEQ ID NO: 2.

The phytase mutant APPAmut8 has an amino acid sequence of SEQ ID NO: 3.

The phytase mutant according to the present invention is the phytase mutant APPAmut9 having phytase activity and comprising the amino acid sequence of SEQ ID NO: 3, except D corresponding to position 133 is replaced with E, R corresponding to position 382 is replaced with I, and S corresponding to position 393 is replaced with I, and wherein the phytase mutant has improved thermal stability as compared to a phytase consisting of the amino acid sequence of SEQ ID NO: 3.

The phytase mutant APPAmut9 has an amino acid sequence of SEQ ID NO: 4. Another aspect of the present invention is provide genes encoding the above mutants.

The gene encoding the said phytase mutant APPAmut5 of the present invention comprises the nucleotide sequence of SEQ ID NO: 5.

The gene encoding the said phytase mutant APPAmut8 of the present invention comprises the nucleotide sequence of SEQ ID NO: 6.

The gene encoding the said phytase mutant APPAmut9 of the present invention comprises the nucleotide sequence of SEQ ID NO: 7.

Another aspect of the present invention is to provide a method of improving the thermal stability of a phytase comprising an amino acid of SEQ ID NO:1, wherein said method comprises the step of
replacing A corresponding to position 57 of SEQ ID NO:1 with C, and A corresponding to position 103 of SEQ ID NO:1 with C, or,
replacing G corresponding to position 101 of SEQ ID NO:1 with C, and V corresponding to position 116 of SEQ ID NO:1 with C, or,
replacing R corresponding to position 271 of SEQ ID NO:1 with C, and E corresponding to position 413 of SEQ ID NO:1 with C, or,
replacing R corresponding to position 353 of SEQ ID NO:1 with C, and L corresponding to position 401 of SEQ ID NO:1 with C, or,
replacing A corresponding to position 147 of SEQ ID NO:1 with C, and Y corresponding to position 268 of SEQ ID NO:1 with C; or
comprising the step of replacing G corresponding to position 65 with R, or,
replacing E corresponding to position 282 of SEQ ID NO: 1 with L, or,
replacing G corresponding to position 365 of SEQ ID NO: 1 with D, or,
replacing D corresponding to position 133 of SEQ ID NO: 1 with E, or,
replacing R corresponding to position 382 of SEQ ID NO: 1 with I, or,
replacing S corresponding to position 393 of SEQ ID NO: 1 with I.

Another aspect of the present invention is to provide a method of improving the thermal stability of a phytase comprising an amino acid of SEQ ID NO:1, wherein said method comprises the steps of replacing A corresponding to position 57 of SEQ ID NO: 1 with C, and A corresponding to position 103 of SEQ ID NO:1 with C,
replacing G corresponding to position 101 of SEQ ID NO:1 with C, and V corresponding to position 116 of SEQ ID NO:1 with C,
replacing R corresponding to position 271 of SEQ ID NO:1 with C, and E corresponding to position 413 of SEQ ID NO:1 with C,
replacing R corresponding to position 353 of SEQ ID NO:1 with C, and L corresponding to position 401 of SEQ ID NO:1 with C, and
replacing A corresponding to position 147 of SEQ ID NO:1 with C, and Y corresponding to position 268 of SEQ ID NO:1 with C.

Another aspect of the present invention is to provide a method of improving the thermal stability of a phytase comprising an amino acid of SEQ ID NO:1, wherein said method comprises the steps of replacing A corresponding to position 57 of SEQ ID NO: 1 with C, and A corresponding to position 103 of SEQ ID NO:1 with C,
replacing G corresponding to position 101 of SEQ ID NO:1 with C, and V corresponding to position 116 of SEQ ID NO:1 with C,
replacing R corresponding to position 271 of SEQ ID NO:1 with C, and E corresponding to position 413 of SEQ ID NO:1 with C,
replacing R corresponding to position 353 of SEQ ID NO:1 with C, and L corresponding to position 401 of SEQ ID NO:1 with C,
replacing A corresponding to position 147 of SEQ ID NO:1 with C, and Y corresponding to position 268 of SEQ ID NO:1 with C,
replacing G corresponding to position 65 with R,
replacing E corresponding to position 282 of SEQ ID NO: 1 with L, and
replacing G corresponding to position 365 of SEQ ID NO: 1 with D.

Another aspect of the present invention is to provide a method of improving the thermal stability of a phytase comprising an amino acid of SEQ ID NO:1, wherein said method comprises the step of replacing A corresponding to position 57 of SEQ ID NO: 1 with C, and A corresponding to position 103 of SEQ ID NO:1 with C,
replacing G corresponding to position 101 of SEQ ID NO:1 with C, and V corresponding to position 116 of SEQ ID NO:1 with C,
replacing R corresponding to position 271 of SEQ ID NO:1 with C, and E corresponding to position 413 of SEQ ID NO:1 with C,
replacing R corresponding to position 353 of SEQ ID NO:1 with C, and L corresponding to position 401 of SEQ ID NO:1 with C,
replacing A corresponding to position 147 of SEQ ID NO:1 with C, and Y corresponding to position 268 of SEQ ID NO:1 with C,
replacing G corresponding to position 65 with R,
replacing E corresponding to position 282 of SEQ ID NO: 1 with L,
replacing G corresponding to position 365 of SEQ ID NO: 1 with D,
replacing D corresponding to position 133 of SEQ ID NO: 1 with E,
replacing R corresponding to position 382 of SEQ ID NO: 1 with I, and
replacing S corresponding to position 393 of SEQ ID NO: 1 with I.

Another aspect of the present invention is to provide a DNA construct comprising the polynucleotide comprising a nucleotide sequence encoding the above phytase mutant.

Another aspect of the present invention is to provide an isolated recombinant cell comprising the above polynucleotide.

Another aspect of the present invention is to provide a method of producing the phytase comprising the steps of transforming an isolated host cell with a DNA construct comprising a polynucleotide which comprises a nucleotide sequence encoding said phytase mutant to obtain a recombinant host cell; cultivating the recombinant host cell to produce the phytase mutant; and recovering the phytase.

The present invention introduces a series of mutations to the phytase APPAmut4, which may involve introducing disulfide bonds, reducing the free energy of unfolding, optimizing the key residues in the coevolution process, and significantly improving the thermal stability of the phytase. Among the mutants of the present invention, the optimal mutant APPAmut9 retains about 70% of its activity after being treated for 5 minutes at 100° C., while the phytase APPAmut4 has already been inactivated. Therefore, the present invention overcomes the shortcomings of the prior art and provides phytase mutants with high thermal stability suitable for wide application in fields such as energy, food, and feed.

### BRIEF DESCRIPTIONS OF THE DRAWINGS

FIG. 1 shows the comparison of the optimal temperatures between the phytase APPAmut4 and its single site and double site mutants;

FIG. 2 shows the comparison of the optimal temperatures between the phytase APPAmut4 and its multi-site mutants.

FIG. 3 shows the comparison of the thermal stability of each single site and double site mutant after being treated at 65° C.;

FIG. 4 shows the comparison of the thermal stability of the phytase APPAmut4 and its multi-site mutants after being treated at 65° C.;

FIG. 5 shows the comparison of t_{1/2} of the phytase APPAmut4 and its mutants.

### EMBODIMENT

### Test Materials and Reagents:

1. Strains and vectors: Pichia pastoris GS115 and expressing vector pPICZαA;
2. Enzymes and other biochemical reagents: Endonucleases;
3. Medium:
(1) Low-salt LB (LLB) for Escherichia coli: 1% peptone, 0.5% yeast extract, 0.5% NaCL, pH natural),
(2) Pichia pastoris YPD medium: 1% yeast extract, 2% peptone, 2% glucose, 7.0;
(3) BMGY medium: 1% yeast extract, 2% peptone, 1% glycerol, 1.34% YNB, 0.00004% Biotin, pH 7.0;
(4) BMMY medium: 1% yeast extract, 2% peptone, 0.5% methanol, 1%, 1.34% YNB, 0.00004% Biotin, pH 7.0.

Suitable biology laboratory methods not particularly mentioned in the examples as below can be found in Sambrook, et al. (Molecular Cloning: A Laboratory Manual. 2nd, ed., Cold Spring Harbor Laboratory, Cold Spring Harbor Laboratory Press, Cold Spring Harbor, NY, 1989), and other kit laboratory manuals.

Method for determining phytase activity: Diluting the enzyme solution with 0.1 mol/L HAc NaAc buffer containing 0.05% BSA and 0.05% Triton X-100 at pH 5.5, adding 100 μL of the diluted enzyme solution to 900 μL of sodium phytate substrate being prepared with 0.1 mol/L HAc NaAc buffer at pH 5.5, reacting at 37° C. for 10 minutes, adding 1 mL of 10% (W/V) TCA to terminate the reaction, adding 1 mL of colorimetric solution containing 1% (W/V) ammonium molybdate tetrahydrate, 3.2% (V/V) concentrated sulfuric acid, 7.32% (W/V) ferrous sulfate for color development, while the control is added with TCA and mixed well before adding the enzyme solution to denature the enzyme, and measuring the OD value under 700 nm light absorption and calculating the enzyme activity.

### Example 1 Site-Directed Mutation of the Phytase APPAmut4

The site directed mutations were performed with the primers as list in the table 1 using the mutagenesis kit.

Firstly, the amino acid sequence of phytase APPAmut4 derived from Yersinia intermedia was performed the mutations of replacing A corresponding to position 57 of SEQ ID NO:1 with C, and A corresponding to position 103 of SEQ ID NO:1 with C to obtain the mutant A57C/A103C, or
replacing G corresponding to position 101 of SEQ ID NO:1 with C, and V corresponding to position 116 of SEQ ID NO:1 with C to to obtain the mutant G101C/V116C, or,
replacing R corresponding to position 271 of SEQ ID NO:1 with C, and E corresponding to position 413 of SEQ ID NO:1 with C to obtain the mutant R271C/E413C, or,
replacing R corresponding to position 353 of SEQ ID NO:1 with C, and L corresponding to position 401 of SEQ ID NO:1 with C to obtain the mutant R353C/L401C, or
replacing A corresponding to position 147 of SEQ ID NO:1 with C, and Y corresponding to position 268 of SEQ ID NO:1 with C to obtain the mutant R353C/L401C.

Secondly, The amino acid sequence of phytase APPAmut4 derived from Yersinia intermedia was performed the mutations of replacing G corresponding to position 65 with R to obtain the mutant G65R, or,
replacing E corresponding to position 282 of SEQ ID NO: 1 with L to obtain the mutant E282L, or,
replacing G corresponding to position 365 of SEQ ID NO: 1 with D to obtain the mutant G365D,
replacing D corresponding to position 133 of SEQ ID NO: 1 with E to obtain the mutant D133E, or
replacing R corresponding to position 382 of SEQ ID NO: 1 with I to obtain the mutant R382I, or,
replacing S corresponding to position 393 of SEQ ID NO: 1 with I to obtain the mutant S393I.

Thirdly, the amino acid sequence of phytase APPAmut4 derived from Yersinia intermedia was performed the mutations of replacing A corresponding to position 57, A corresponding to position 103, G corresponding to position 101, V corresponding to position 116, R corresponding to position 271, E corresponding to position 413, R corresponding to position 353, L corresponding to position 401, A corresponding to position 147, Y corresponding to position 268 of SEQ ID NO: 1 with C respectively, to obtain the mutant APPAmut5.

Fourthly, the amino acid sequence of the APPAmut5 was performed the additional mutations of replacing G corresponding to position 65 with R, E corresponding to position 282 with L, and G corresponding to position 365 with D, using the primers for the site directed mutagenesis, to obtain the mutant APPAmut8.

Fifthly, the amino acid sequence of the APPAmut8 was performed the additional mutations of replacing D corresponding to position 133 with E, R corresponding to position 382 with I, and S corresponding to position 393 with I using the primers for the site directed mutagenesis to obtain the mutant APPAmut9.

TABLE 2
primers for the site directed mutagenesis

| Primer | Sequence of the primers (5′-3′) |
|---|---|
| A147C-F | ACCCACAAATGTGTTGAAGAGAG |
| Y268C-R | TCTAGCAATACAAGGAGTCTTTG |
| R271C-F | TACATTGCTTGTCACAAAGGAACTC |
| E413C-R | ACAAGCTGGACATATGGCCTGAGCAACC |
| G101C-R | TCCTGGAGCGATACCATCCAAGAATGCTTGACAGGTCA |
| | AACGAGTTC |
| V116C-F | TTCTTGGATGGTATCGCTCCAGGATGTGGCTTGAAATG |
| | TCACTACCAGG |
| A57C-F | CACCAAGAGGTTGTCAGTTGGTTAC |
| A103C-R | CATCCAAGAAACATTGTCCGGTCAAAC |
| R353C-F | CAACCACCAGTGTTACGTTGCTGTAAAG |
| L401C-R | GAAAGTATCACACTGGCACAAC |
| R353C-R | AGCAACGTAACACTGGTGGTTGTCAGGGTTTTGC |
| L401C-F | GCCAGTGTGATACTTTCCAGAAGAAGG |
| G365D-F | CAGACTATGGATCAATTGCGTAACGCAGAG |
| G365D-R | CGCAATTGATCCATAGTCTGATAGAACAAC |
| A89V-F | GACATCTACGTTCAAGCTGACGTTGATCAAAG |
| A89V-R | GTCAGCTTGAACGTAGATGTCAGCATCAGTAG |
| G65R-F | ATGGGTAGATTCTACGGTGACTATTTCAG |
| G65R-R | ACCGTAGAATCTACCCATCAAAGTAACCAAC |
| E282L-F | CAGCAAATCTTGACTGCTTTGGTCCTCCAAAG |
| E282L-R | CAAAGCAGTCAAGATTTGCTGAAGCAAGGGAG |
| D133E-F | CACCCTGTTGAAGCAGGTGTTTGTAAGCTTG |
| D133E-R | AAACACCTGCTTCAACAGGGTGGAACAGTGG |
| R382I-F | CCAGCCGGTATTGTTCCTGTCGCAATTGACG |
| R382I-R | GACAGGAACAATACCGGCTGGGTTGTTCTTC |
| S393I-F | TGTGAGAACATTGGAGATGACAAGTTGTGCC |
| S393I-R | GTCATCTCCAATGTTCTCACAACCGTCAATTG |

### Example 2 Constructing the Strain Expressing the Phytase APPAmut4 and its Mutants

PCR amplification was performed using primers containing the corresponding mutation sites and plasmid pPICZαA-appamut4 as the template, and then the PCR amplification product was analyzed by 1% agarose gel electrophoresis, wherein If the size of the band was consistent with the theoretical value, the PCR reaction was successful in obtaining the target product. In order to eliminate the interference of template plasmids on subsequent experiments, 1 μL of restriction enzyme Dpn I was added to the PCR system based on the methylation difference between template plasmids and PCR products, followed by being digested at 37° C. for 1 to 2 hours. Then, 10 μL of the product was transformed into E. coli DMT competent cells, followed by extracting the recombinant plasmid when the result of sequencing is correct, linearizing it using the restriction enzyme Pme I, purifying and recovering the product, and transforming it into the competent cells of Pichia pastoris GS115 to obtain the recombinant expression strain of Pichia pastoris.

### Example 3 Preparing the Phytase APPAmut4 and its Mutant

The obtained recombinant expression strain was inoculated into YPD medium for seed culture at 200 rpm and 30° C. for 48 hours, followed by being transferred to BMGY medium in 1% inoculation amount and cultured at 200 rpm and 30° C. for 48 hours. Then, the supernatant was discard after being centrifuged at 4500 rpm for 5 minutes, the bacterial cells were collected, and BMMY medium containing 0.5% of methanol was added for inducing the expression with a addition of 0.5% of methanol every 12 hours for a total of 48 hours of induction.

The induced bacterial solution was centrifuged at 12000 rpm for 10 minutes, to collect the supernatant for concentration, followed by being dialyzed with 20 mM of Tris HCl at pH 8.0. Then, the dialyzed enzyme solution was subjected to anion exchange chromatography, with the solution A containing 20 mM of Tris HCL at pH 8.0 and the solution B obtained by adding 1 M of NaCL to the solution A, to purify the protein, and the eluent was collected for SDS-PAGE analysis.

### Example 4 Measuring the Enzymatic Properties of Mutants

### (1) Determining the Optimal Temperature

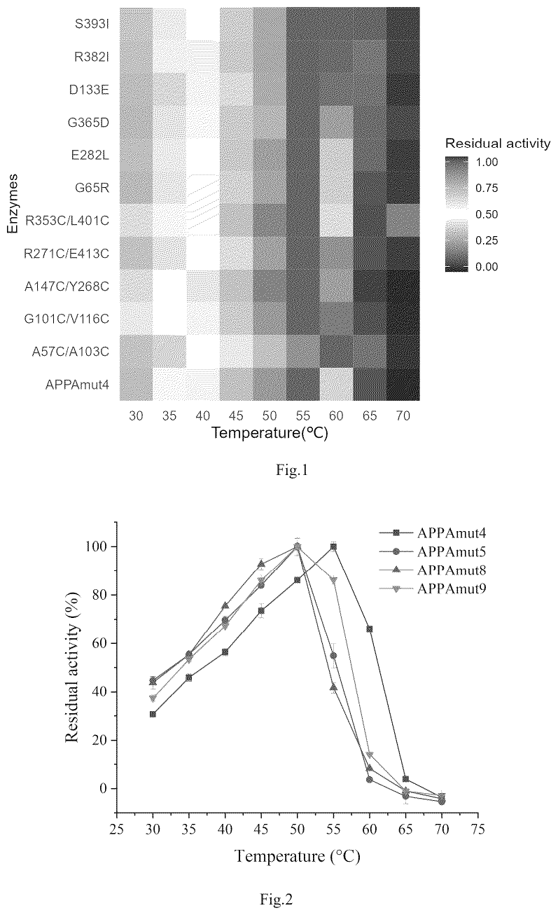

The enzyme activity of wild-type and mutant was measured at the different temperatures of 30° C., 35° C., 40° C., 45° C., 50° C., 55° C. C, 60° C., 65° C., and 70° C. with 0.1 mol/L of HAc NaAc buffer at pH to determine the optimal temperature, wherein the activity corresponding to the optimal temperature was defined as 100%, and the remaining enzyme activity at the other temperatures was calculated. As shown in FIG. 1, the optimal temperature for APPAmut4 is 55° C., while the optimal temperature for mutant A57C/A103C increases to 60° C., the optimal temperature for L253C/P331C decreases to 50° C., and the other mutants remain unchanged. However, the enzyme activity of the mutants D133E, R382I, and S393I at 60° C. is almost the same as that at 55° C., indicating that all three mutants have good catalytic function between 55 and 60° C. As shown in FIG. 2, the optimal temperature for APPAmuT5, APPAmuT8, and APPAmuT9 is 50° C.

### (2) Measuring the Thermal Stability

The purified protein was diluted with 0.1 mol/L of HAc NaAc buffer in pH 5.5 containing 0.05% BSA and 0.05% Triton X-100 to an appropriate multiple, followed by taking 100 μL for being incubated at 65° C. for 0, 2, 5, 10, 15, and 30 min respectively, and then measuring the corresponding enzyme activity, wherein the activity at 0 min is considered as 100%, and the remaining enzyme activities at different incubation time were calculated respectively.

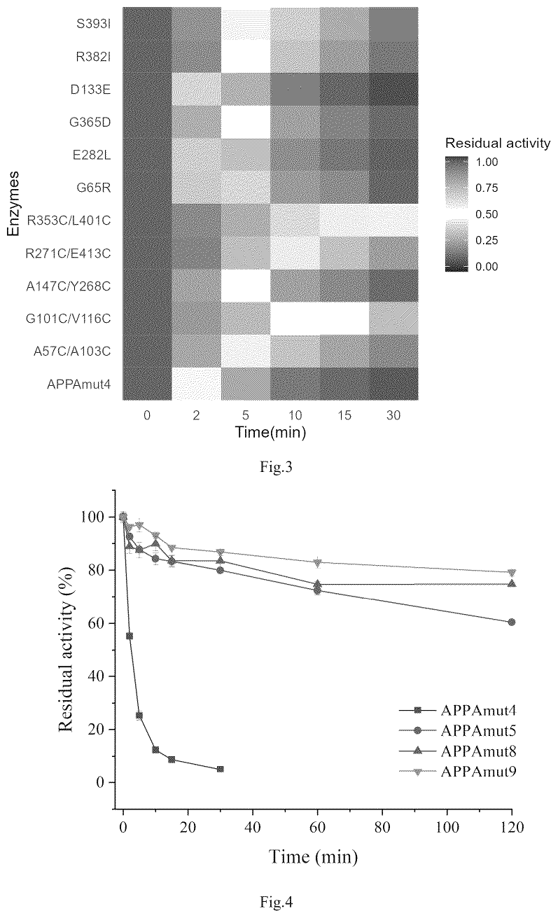

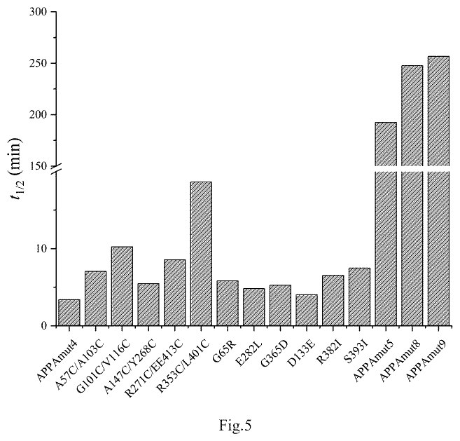

As shown in FIG. 3, after being treated at 65° C. for 10 minutes, the remaining enzyme activity of APPAmut4 was 12.3%, while the remaining enzyme activity of each double point and single point mutant ranged from 13.6% to 64.4%; As shown in FIG. 4, the residual enzyme activities of the mutants APPAmut5, APPAmut8, and APPAmut9 after being treated at 65° C. for 10 minutes were 84.3%, 89.9%, and 93.2%, respectively.

Half life t_{1/2} refers to the time that takes for the initial activity to decrease by 50% at a given temperature, and was calculated by the following formula,

$${t}_{1 / 2} = \frac{ln 2}{{k}_{d}} ,$$

wherein k_{d} is the deactivation rate constant, which can be obtained through the following linear regression:

$$ln \frac{{A}_{t}}{{A}_{0}} = - {k}_{d} t ,$$

wherein Aₜ refers to the residual activity, A₀ is the initial activity, and t is the processing time at the studied temperatures.

As shown in FIG. 5, the value of t_{1/2} of the phytaset APPAmut4 at 65° C. is 3.4 min, while the values of t_{1/2} of each double point and single point mutant are ranging from 4.0 to 18.6 min, increased by from 0.6 to 15.2 min; and the values of t_{1/2} of the combined mutants APPAmut5, APPAmut8, and APPAmut9 at 65° C. were 192.5, 247.6, and 256.7 min, respectively, which were 56, 72, and 74 times higher than that of the phytase APPAmut4, respectively. In practical applications, the life of enzyme is significantly correlated with its kinetic stability, i.e. the time or temperature required for proteins to maintain partial activity during irreversible denaturation. The half-life t_{1/2} is a conventional parameter for characterizing the kinetic stability of enzymes, and the larger its value, the higher the kinetic stability of the enzyme. The test measurements showed that the value of t_{1/2} of the mutant increased to the varying degrees as compared to the phytase APPAmut4 through experimental measurements, indicating that its thermal stability was improved to varying degrees. The value of t_{1/2} of the mutant APPAmut9 is as high as 256.7 minutes, indicating a significant improvement in thermal stability.

### (3) Determination of Dynamic Parameters

The different concentrations of sodium phytate ranging from 0.05 to 1.00 mM were prepared as substrates and the activity of phytase was measured at 37° C. and pH 5.5, wherein the software GraphPad Prism was used for data processing, and the values of Kₘ and k_{cat} were calculated. As shown in Table 2, the value of Kₘ of the phytase APPAmut4 is 0.14 mM, while the values of Kₘ of the mutants A57C/A103C, G101C/V16C, A147C/Y268C, R271C/E413C, R353C/L401C, and G65R have increased in the varying degrees ranging from 0.18 to 0.22, indicating a decrease in substrate affinity. The catalytic efficiency k_{cat}/Kₘ of the phytase APPAmut4 was 12322/mM/s, while the k_{cat}/Kₘ of the mutants A147C/Y268C and G65R decreased to 10823 and 9009/mM/s, respectively, indicating that the mutation affected its catalytic activity, and the catalytic efficiency of the remaining single point and double point mutants remains unchanged or slightly improves. The k_{cat}/Kₘ for the combination mutants APPAmut5, APPAmut8, and APPAmut9 are 11632, 13018, and 11537/mM/s, which remained relatively unchanged as compared to the phytase APPAmut4, respectively, indicating that a significant the improvement in thermal stability didn't reduce their catalytic efficiency.

TABLE 2
Dynamics parameters of the phytase APPAmut4 and its mutants

| Enzymes | Kₘ (mM) | Vₘₐₓ (μmol/min/mg) | k_{cat} (/s) | k_{cat}/Kₘ (/mM/s) |
|---|---|---|---|---|
| APPAmut4 | 0.14 ± 0.03 | 2272 ± 95 | 1725 ± 72 | 12322 ± 1577 |
| A57C/A103C | 0.18 ± 0.03 | 2845 ± 171 | 2160 ± 130 | 12068 ± 1368 |
| G101C/V116C | 0.20 ± 0.03 | 2932 ± 157 | 2226 ± 119 | 11131 ± 1132 |
| A147C/Y268C | 0.22 ± 0.03 | 3136 ± 163 | 2381 ± 124 | 10823 ± 1019 |
| R271C/E413C | 0.18 ± 0.03 | 3177 ± 203 | 2412 ± 154 | 13402 ± 1545 |
| R353C/L401C | 0.20 ± 0.02 | 3400 ± 163 | 2581 ± 124 | 12908 ± 955 |
| G65R | 0.22 ± 0.04 | 2634 ± 101 | 2000 ± 77 | 9009 ± 992 |
| E282L | 0.15 ± 0.02 | 2300 ± 88 | 1746 ± 67 | 11269 ± 967 |
| G365D | 0.14 ± 0.02 | 2411 ± 76 | 1831 ± 58 | 13158 ± 1148 |
| D133E | 0.13 ± 0.03 | 2477 ± 76 | 1881 ± 58 | 14989 ± 1960 |
| R382I | 0.16 ± 0.02 | 2564 ± 106 | 1947 ± 80 | 11958 ± 993 |
| S393I | 0.14 ± 0.03 | 2772 ± 126 | 2105 ± 96 | 14716 ± 1912 |
| APPAmut5 | 0.15 ± 0.02 | 2298 ± 108 | 1744 ± 82 | 11632 ± 1048 |
| APPAmut8 | 0.15 ± 0.02 | 2630 ± 127 | 1997 ± 96 | 13018 ± 1181 |
| APPAmut9 | 0.15 ± 0.02 | 2279 ± 134 | 1730 ± 102 | 11537 ± 1109. |

The above embodiments are only used to illustrate the technical solution of the present application and do not limit the scope of protection of the present application.

1. A phytase mutant having phytase activity and comprising the amino acid sequence of SEQ ID NO:1, except A corresponding to position 57 and A corresponding to position 103 of SEQ ID NO: 1 are replaced with C respectively, or,
G corresponding to position 101 and V corresponding to position 116 of SEQ ID NO: 1 are replaced with C respectively, or
R corresponding to position 271 and E corresponding to position 413 of SEQ ID NO: 1 are replaced with C respectively, or
R corresponding to position 353 and L corresponding to position 401 of SEQ ID NO: 1 are replaced with C respectively, or
A corresponding to position 147 and Y corresponding to position 268 of SEQ ID NO: 1 are replaced with C respectively, and wherein the phytase mutant has the improved thermal stability as compared to a phytase consisting of the amino acid sequence of SEQ ID NO: 1.

2. The phytase mutant according to claim 1, wherein said phytase mutant comprises the amino acid sequence of SEQ ID NO:1, further except that G corresponding to position 65 is replaced with R, or,
E corresponding to position 282 of SEQ ID NO: 1 is replaced with L or,
G corresponding to position 365 of SEQ ID NO: 1 is replaced with D, or
D corresponding to position 133 of SEQ ID NO: 1 is replaced with E, or
R corresponding to position 382 of SEQ ID NO: 1 is replaced with I, or,
S corresponding to position 393 of SEQ ID NO: 1 is replaced with I.

3. The phytase mutant according to claim 1, wherein said phytase mutant comprises the amino acid sequence of SEQ ID NO:2, or SEQ ID NO:3, or SEQ ID NO: 4.

4. A polynucleotide comprising a nucleotide sequence encoding the phytase mutant of claim 1.

5. A DNA construct comprising the polynucleotide of claim 4.

6. An isolated recombinant cell comprising the polynucleotide of claim 4.

7. A method of improving the thermal stability of a phytase comprising an amino acid of SEQ ID NO:1, wherein said method comprising the step of
replacing A corresponding to position 57 of SEQ ID NO:1 with C, and A corresponding to position 103 of SEQ ID NO:1 with C, or,
replacing G corresponding to position 101 of SEQ ID NO:1 with C, and V corresponding to position 116 of SEQ ID NO:1 with C, or,
replacing R corresponding to position 271 of SEQ ID NO:1 with C, and E corresponding to position 413 of SEQ ID NO:1 with C, or,
replacing R corresponding to position 353 of SEQ ID NO: 1 with C, and L corresponding to position 401 of SEQ ID NO:1 with C, or,
replacing A corresponding to position 147 of SEQ ID NO:1 with C, and Y corresponding to position 268 of SEQ ID NO:1 with C.

8. The method according to claim 7, wherein said method further comprises the step of replacing G corresponding to position 65 with R, or,
replacing E corresponding to position 282 of SEQ ID NO: 1 with L, or,
replacing G corresponding to position 365 of SEQ ID NO: 1 with D, or,
replacing D corresponding to position 133 of SEQ ID NO: 1 with E, or,
replacing R corresponding to position 382 of SEQ ID NO: 1 with I, or,
replacing S corresponding to position 393 of SEQ ID NO: 1 with I.

9. The method according to claim 7, wherein said method comprises the step of replacing A corresponding to position 57, A corresponding to position 103, G corresponding to position 101, V corresponding to position 116, R corresponding to position 271, E corresponding to position 413, R corresponding to position 353, L corresponding to position 401, A corresponding to position 147, and Y corresponding to position 268 of SEQ ID NO:1 with C, respectively.

10. The method according to claim 9, wherein said method further comprises the steps of replacing G corresponding to position 65 with R, replacing E corresponding to position 282 of SEQ ID NO: 1 with L, and replacing G corresponding to position 365 of SEQ ID NO: 1 with D.

11. The method according to claim 10, wherein said method further comprises the steps of replacing D corresponding to position 133 of SEQ ID NO: 1 with E, replacing R corresponding to position 382 of SEQ ID NO: 1 with I, and replacing S corresponding to position 393 of SEQ ID NO: 1 with I.

12. A use of the phytase mutant of claim 1 to hydrolyzing the phytic acid.


SEQUENCE LISTING (33 sequences)

SEQ ID NO: 1 - protein, 418 aa, synthetic construct
AAPVAIQPTGYTLERVVILSRHGVRSPTKQTQLMNDVTPDTWPQWPVAAGELTPRGAQLVTLMGGFYGDYFRSQGLLAAGCPTDADIYAQADVDQRTRLTGQAFLDGIAPGCGLKVHYQADLKKVDPLFHPVDAGVCKLDSTQTHKAVEERLGGPLSELSKRYAKPFAQMGEILNFAASPYCKSLQQQGKTCDFANFAANKITVNKPGTKVSLSGPLALSSTLGEIFLLQNSQAMPDVAWHRLTGEDNWISLLSLHNAQFDLMAKTPYIARHKGTPLLQQIETALVLQRDAQGQTLPLSPQTKILFLGGHDTNIANIAGMLGLNWQLPQQPDNTPPGGGLVFELWQNPDNHQRYVAVKLFYQTMGQLRNAEKLDLKNNPAGRVPVAIDGCENSGDDKLCQLDTFQKKVAQAIEPACHI

SEQ ID NO: 2 - protein, 418 aa, synthetic construct
AAPVAIQPTGYTLERVVILSRHGVRSPTKQTQLMNDVTPDTWPQWPVAAGELTPRGCQLVTLMGGFYGDYFRSQGLLAAGCPTDADIYAQADVDQRTRLTCQCFLDGIAPGCGLKCHYQADLKKVDPLFHPVDAGVCKLDSTQTHKCVEERLGGPLSELSKRYAKPFAQMGEILNFAASPYCKSLQQQGKTCDFANFAANKITVNKPGTKVSLSGPLALSSTLGEIFLLQNSQAMPDVAWHRLTGEDNWISLLSLHNAQFDLMAKTPCIACHKGTPLLQQIETALVLQRDAQGQTLPLSPQTKILFLGGHDTNIANIAGMLGLNWQLPQQPDNTPPGGGLVFELWQNPDNHQCYVAVKLFYQTMGQLRNAEKLDLKNNPAGRVPVAIDGCENSGDDKLCQCDTFQKKVAQAICPACHI

SEQ ID NO: 3 - protein, 418 aa, synthetic construct
AAPVAIQPTGYTLERVVILSRHGVRSPTKQTQLMNDVTPDTWPQWPVAAGELTPRGCQLVTLMGRFYGDYFRSQGLLAAGCPTDADIYAQADVDQRTRLTCQCFLDGIAPGCGLKCHYQADLKKVDPLFHPVDAGVCKLDSTQTHKCVEERLGGPLSELSKRYAKPFAQMGEILNFAASPYCKSLQQQGKTCDFANFAANKITVNKPGTKVSLSGPLALSSTLGEIFLLQNSQAMPDVAWHRLTGEDNWISLLSLHNAQFDLMAKTPCIACHKGTPLLQQILTALVLQRDAQGQTLPLSPQTKILFLGGHDTNIANIAGMLGLNWQLPQQPDNTPPGGGLVFELWQNPDNHQCYVAVKLFYQTMDQLRNAEKLDLKNNPAGRVPVAIDGCENSGDDKLCQCDTFQKKVAQAICPACHI

SEQ ID NO: 4 - protein, 418 aa, synthetic construct
AAPVAIQPTGYTLERVVILSRHGVRSPTKQTQLMNDVTPDTWPQWPVAAGELTPRGCQLVTLMGRFYGDYFRSQGLLAAGCPTDADIYAQADVDQRTRLTCQCFLDGIAPGCGLKCHYQADLKKVDPLFHPVEAGVCKLDSTQTHKCVEERLGGPLSELSKRYAKPFAQMGEILNFAASPYCKSLQQQGKTCDFANFAANKITVNKPGTKVSLSGPLALSSTLGEIFLLQNSQAMPDVAWHRLTGEDNWISLLSLHNAQFDLMAKTPCIACHKGTPLLQQILTALVLQRDAQGQTLPLSPQTKILFLGGHDTNIANIAGMLGLNWQLPQQPDNTPPGGGLVFELWQNPDNHQCYVAVKLFYQTMDQLRNAEKLDLKNNPAGIVPVAIDGCENIGDDKLCQCDTFQKKVAQAICPACHI

SEQ ID NO: 5 - DNA, 1258 bp, synthetic construct
  source 1..1258: mol_type = other DNA
gctgctccagtcgctatccaacctactggttacactcttgagagagttgtcatcttgtctagacatggtgttagatccccaactaagcagacccaattgatgaacgatgtgacacctgacacgtggcctcaatggccagttgcagctggtgagttgacaccaagaggttgtcagttggttactttgatgggtggattctacggtgactatttcagatcccaaggattgcttgctgccggctgtcctactgatgctgacatctacgcacaagctgacgttgatcaaagaactcgtttgacctgtcaatgtttcttggatggtatcgctccaggatgtggcttgaaatgtcactaccaggctgatttgaagaaggttgatccactgttccaccctgttgatgcaggtgtttgtaagcttgactctactcaaacccacaaatgtgttgaagagagattgggtggtccattgagcgaactttcgaagagatacgccaaaccttttgcacaaatgggagagatcctgaacttcgcagcgtcaccttactgtaagagtttgcaacagcaaggtaagacttgcgactttgccaacttcgctgccaacaagatcactgtcaacaagcctggaacgaaagtatccttgtctggtccattggctctgtcttccactcttggagaaatcttcttgctgcaaaactctcaagctatgccagatgttgcctggcacagattgaccggtgaggacaactggatttctttgctctccttacacaatgcccaattcgatctgatggcaaagactccttgtattgcttgtcacaaaggaactcccttgcttcagcaaatcgaaactgctttggtcctccaaagggacgcccagggtcaaactttgccattgtctcctcagaccaagatcctgttcttgggtggacacgatactaacatcgcaaacatcgctgggatgttgggtttgaactggcaacttccacagcaaccagacaacaccccacctggcggtggtctagtcttcgagttgtggcaaaaccctgacaaccaccagtgttacgttgctgtaaagttgttctatcagactatgggacaattgcgtaacgcagagaagttggatttgaagaacaacccagccggtagggttcctgtcgcaattgacggttgtgagaactctggagatgacaagttgtgccagtgtgatactttccagaagaaggttgctcaggccatattgtccagcttgtcacatctaa

SEQ ID NO: 6 - DNA, 1258 bp, synthetic construct
  source 1..1258: mol_type = other DNA
gctgctccagtcgctatccaacctactggttacactcttgagagagttgtcatcttgtctagacatggtgttagatccccaactaagcagacccaattgatgaacgatgtgacacctgacacgtggcctcaatggccagttgcagctggtgagttgacaccaagaggttgtcagttggttactttgatgggtagattctacggtgactatttcagatcccaaggattgcttgctgccggctgtcctactgatgctgacatctacgcacaagctgacgttgatcaaagaactcgtttgacctgtcaatgtttcttggatggtatcgctccaggatgtggcttgaaatgtcactaccaggctgatttgaagaaggttgatccactgttccaccctgttgatgcaggtgtttgtaagcttgactctactcaaacccacaaatgtgttgaagagagattgggtggtccattgagcgaactttcgaagagatacgccaaaccttttgcacaaatgggagagatcctgaacttcgcagcgtcaccttactgtaagagtttgcaacagcaaggtaagacttgcgactttgccaacttcgctgccaacaagatcactgtcaacaagcctggaacgaaagtatccttgtctggtccattggctctgtcttccactcttggagaaatcttcttgctgcaaaactctcaagctatgccagatgttgcctggcacagattgaccggtgaggacaactggatttctttgctctccttacacaatgcccaattcgatctgatggcaaagactccttgtattgcttgtcacaaaggaactcccttgcttcagcaaatcttgactgctttggtcctccaaagggacgcccagggtcaaactttgccattgtctcctcagaccaagatcctgttcttgggtggacacgatactaacatcgcaaacatcgctgggatgttgggtttgaactggcaacttccacagcaaccagacaacaccccacctggcggtggtctagtcttcgagttgtggcaaaaccctgacaaccaccagtgttacgttgctgtaaagttgttctatcagactatggatcaattgcgtaacgcagagaagttggatttgaagaacaacccagccggtagggttcctgtcgcaattgacggttgtgagaactctggagatgacaagttgtgccagtgtgatactttccagaagaaggttgctcaggccatattgtccagcttgtcacatctaa

SEQ ID NO: 7 - DNA, 1258 bp, synthetic construct
  source 1..1258: mol_type = other DNA
gctgctccagtcgctatccaacctactggttacactcttgagagagttgtcatcttgtctagacatggtgttagatccccaactaagcagacccaattgatgaacgatgtgacacctgacacgtggcctcaatggccagttgcagctggtgagttgacaccaagaggttgtcagttggttactttgatgggtagattctacggtgactatttcagatcccaaggattgcttgctgccggctgtcctactgatgctgacatctacgcacaagctgacgttgatcaaagaactcgtttgacctgtcaatgtttcttggatggtatcgctccaggatgtggcttgaaatgtcactaccaggctgatttgaagaaggttgatccactgttccaccctgttgaagcaggtgtttgtaagcttgactctactcaaacccacaaatgtgttgaagagagattgggtggtccattgagcgaactttcgaagagatacgccaaaccttttgcacaaatgggagagatcctgaacttcgcagcgtcaccttactgtaagagtttgcaacagcaaggtaagacttgcgactttgccaacttcgctgccaacaagatcactgtcaacaagcctggaacgaaagtatccttgtctggtccattggctctgtcttccactcttggagaaatcttcttgctgcaaaactctcaagctatgccagatgttgcctggcacagattgaccggtgaggacaactggatttctttgctctccttacacaatgcccaattcgatctgatggcaaagactccttgtattgcttgtcacaaaggaactcccttgcttcagcaaatcttgactgctttggtcctccaaagggacgcccagggtcaaactttgccattgtctcctcagaccaagatcctgttcttgggtggacacgatactaacatcgcaaacatcgctgggatgttgggtttgaactggcaacttccacagcaaccagacaacaccccacctggcggtggtctagtcttcgagttgtggcaaaaccctgacaaccaccagtgttacgttgctgtaaagttgttctatcagactatggatcaattgcgtaacgcagagaagttggatttgaagaacaacccagccggtattgttcctgtcgcaattgacggttgtgagaacattggagatgacaagttgtgccagtgtgatactttccagaagaaggttgctcaggccatattgtccagcttgtcacatctaa

SEQ ID NO: 8 - DNA, 23 bp, synthetic construct
  source 1..23: mol_type = other DNA
acccacaaatgtgttgaagagag

SEQ ID NO: 9 - DNA, 23 bp, synthetic construct
  source 1..23: mol_type = other DNA
tctagcaatacaaggagtctttg

SEQ ID NO: 10 - DNA, 25 bp, synthetic construct
  source 1..25: mol_type = other DNA
tacattgcttgtcacaaaggaactc

SEQ ID NO: 11 - DNA, 28 bp, synthetic construct
  source 1..28: mol_type = other DNA
acaagctggacatatggcctgagcaacc

SEQ ID NO: 12 - DNA, 47 bp, synthetic construct
  source 1..47: mol_type = other DNA
tcctggagcgataccatccaagaatgcttgacaggtcaaacgagttc

SEQ ID NO: 13 - DNA, 49 bp, synthetic construct
  source 1..49: mol_type = other DNA
ttcttggatggtatcgctccaggatgtggcttgaaatgtcactaccagg

SEQ ID NO: 14 - DNA, 25 bp, synthetic construct
  source 1..25: mol_type = other DNA
caccaagaggttgtcagttggttac

SEQ ID NO: 15 - DNA, 27 bp, synthetic construct
  source 1..27: mol_type = other DNA
catccaagaaacattgtccggtcaaac

SEQ ID NO: 16 - DNA, 28 bp, synthetic construct
  source 1..28: mol_type = other DNA
caaccaccagtgttacgttgctgtaaag

SEQ ID NO: 17 - DNA, 22 bp, synthetic construct
  source 1..22: mol_type = other DNA
gaaagtatcacactggcacaac

SEQ ID NO: 18 - DNA, 34 bp, synthetic construct
  source 1..34: mol_type = other DNA
agcaacgtaacactggtggttgtcagggttttgc

SEQ ID NO: 19 - DNA, 27 bp, synthetic construct
  source 1..27: mol_type = other DNA
gccagtgtgatactttccagaagaagg

SEQ ID NO: 20 - DNA, 30 bp, synthetic construct
  source 1..30: mol_type = other DNA
cagactatggatcaattgcgtaacgcagag

SEQ ID NO: 21 - DNA, 30 bp, synthetic construct
  source 1..30: mol_type = other DNA
cgcaattgatccatagtctgatagaacaac

SEQ ID NO: 22 - DNA, 32 bp, synthetic construct
  source 1..32: mol_type = other DNA
gacatctacgttcaagctgacgttgatcaaag

SEQ ID NO: 23 - DNA, 32 bp, synthetic construct
  source 1..32: mol_type = other DNA
gtcagcttgaacgtagatgtcagcatcagtag

SEQ ID NO: 24 - DNA, 29 bp, synthetic construct
  source 1..29: mol_type = other DNA
atgggtagattctacggtgactatttcag

SEQ ID NO: 25 - DNA, 31 bp, synthetic construct
  source 1..31: mol_type = other DNA
accgtagaatctacccatcaaagtaaccaac

SEQ ID NO: 26 - DNA, 32 bp, synthetic construct
  source 1..32: mol_type = other DNA
cagcaaatcttgactgctttggtcctccaaag

SEQ ID NO: 27 - DNA, 32 bp, synthetic construct
  source 1..32: mol_type = other DNA
caaagcagtcaagatttgctgaagcaagggag

SEQ ID NO: 28 - DNA, 31 bp, synthetic construct
  source 1..31: mol_type = other DNA
caccctgttgaagcaggtgtttgtaagcttg

SEQ ID NO: 29 - DNA, 31 bp, synthetic construct
  source 1..31: mol_type = other DNA
aaacacctgcttcaacagggtggaacagtgg

SEQ ID NO: 30 - DNA, 31 bp, synthetic construct
  source 1..31: mol_type = other DNA
ccagccggtattgttcctgtcgcaattgacg

SEQ ID NO: 31 - DNA, 31 bp, synthetic construct
  source 1..31: mol_type = other DNA
gacaggaacaataccggctgggttgttcttc

SEQ ID NO: 32 - DNA, 31 bp, synthetic construct
  source 1..31: mol_type = other DNA
tgtgagaacattggagatgacaagttgtgcc

SEQ ID NO: 33 - DNA, 32 bp, synthetic construct
  source 1..32: mol_type = other DNA
gtcatctccaatgttctcacaaccgtcaattg